(19) United States
(12) Patent Application Publication
(10) Pub. No.: US 2024/0118265 A1
(43) Pub. Date: Apr. 11, 2024

(54) MATERIALS AND METHODS TO IMPROVE IN VITRO TOXICITY PREDICTIONS

(71) Applicants: Stewart Lebrun, Anaheim, CA (US); Linda Nguyen, Orange, CA (US)

(72) Inventors: Stewart Lebrun, Anaheim, CA (US); Linda Nguyen, Orange, CA (US)

(21) Appl. No.: 18/481,774

(22) Filed: Oct. 5, 2023

Related U.S. Application Data
(60) Provisional application No. 63/414,670, filed on Oct. 10, 2022.

(51) Int. Cl. G01N 33/50

(52) U.S. Cl. CPC G01N 33/5014

(57) ABSTRACT

The disclosure relates to materials and methods for the in vitro testing of irritants. It was discovered that the accuracy of nonanimal toxicity tests is improved when the distance migrated on a synthetic substrate is used as a correction factor for macromolecular, cell, and specific organotypic eye toxicity test systems. The method involves applying the test substance to a solid support test material, measuring the migration distance and then using the migration distance measurement to improve on an existing nonanimal toxicity test; where the existing toxicity test prediction can be: (1) a macromolecular test, (2) a cultured epithelium-based (3) and/or an organotypic test; measuring a test system response; and multiplying, adding, or otherwise using as a correction factor, the measured migration distance, and using this to have an improved prediction of the toxicity of the test substance based on the combined response.

### BACKGROUND

Most chemical safety testing for the eye has traditionally been performed using the method of Draize (Draize et al., 1944), as modified by Kay and Calandra (Kay and Calandra, 1962). This now-controversial procedure involves instilling the substance under evaluation within the conjunctival sac of a New Zealand White rabbit. Indices of toxicity are recorded for the cornea, iris, and conjunctiva at regular time intervals for up to 21 days.

Draize test data have traditionally been used to derive a numerical score of ocular irritation; however, modern classification systems use the same data with a slightly different statistical treatment to develop an irritation category. Modern ocular irritation classification schemes include the European Union (EU), Globally Harmonized System of classification and labeling of chemicals (GHS), and Environmental Protection Agency (EPA) systems, which are not harmonized with one another. The EU Dangerous Substance Directive (DSD) classification and labeling system does not include cosmetics; it was applied in accordance with the Commission Directive 2001/59/EC and includes categories R41 (risk of serious damage to the eye) and R36 (ocular irritant) (EC, 2001). DSD was replaced by the Classification, Labeling, and Packaging (CLP) regulation aligned with GHS (EC, 2008a). The GHS system includes classes NC (not classified as an irritant), 2A (reversal by 7 days), 2B (reversal by 14 days), and 1 (no reversal by 21 days) (EC, 2008b; UN, 2011). GHS classification is used to satisfy U.S. Food and Drug Administration and international safety labeling requirements and plays an important role in commercial product liability and consumer product satisfaction. Guidance documents produced by the Organization for Economic Trade and Development (OECD) are available to coordinate international trade. The OECD describes the standard rabbit eye test (the gold standard for GHS eye safety classification), which is required for safety data sheet documentation accompanying hazardous chemicals and products.

The EPA classification includes classes I (corrosive), II (moderate irritant), III (mild irritant), and IV (nonirritant), in accordance with the guidelines in the Label Review Manual (US EPA, 2003) and based on test methods described in the Acute Eye Irritation Health Effects Test Guideline (US EPA, 1998). Corrosives result in irreversible damage to the eye, whereas ocular irritation is reversible. EPA class III includes irritation at 24 h. The GHS classification system, now widely accepted in the EU, does not include a category with a comparable short-duration sensitivity limitation. The EPA classification system is required for agrochemicals and other registrations and has commercial significance, especially for cosmetics and personal care products used around the eye.

One of the needs for nonanimal safety tests originated from bans or pending bans on the use of animals for the safety of cosmetics and other products. The EU banned animal testing of finished cosmetic products in 2004, animal-tested ingredients 4 years later, and the transport and sale of cosmetics containing ingredients tested on animals in 2013, pledging to push other parts of the world to accept alternatives (Kanter, 2017). As of 2014, there are bans or severe limitations in Norway, Israel, India, and Brazil (Senate Joint Resolution 22, 2014), and by 2017, the list of countries had grown to 37, according to the Humane Society of the U.S. (Humane Society, 2017).

The United States has been slow to ban animal testing or mandate the use of nonanimal alternatives in the product testing industry; however, recent legislation will ban animals for a wide range of testing applications that have traditionally used live animals. Bill H.R.2790 “The Humane Cosmetics Act” was introduced on Jun. 6, 2017 and would prohibit animal testing of cosmetics within 1 year and the sale or transport of cosmetics tested on animals within 3 years after enactment, which is now supported by more than 200 cosmetics companies and stakeholders (H.R.4148, 2014). Additionally, the “Frank R. Lautenberg Chemical Safety for the 21st Century Act”—S.697, which revises the Toxic Substances Control Act of 1976 (TSCA)—was passed on Jun. 22, 2016. The TSCA now requires EPA to evaluate existing and new chemicals to determine whether regulatory control of a certain chemical is warranted and if it presents an unreasonable risk of injury to health or the environment so as to reduce risks to a reasonable level. The law also requires EPA to “reduce and replace, to the extent practical . . . the use of vertebrate animals in testing chemicals to provide information of equivalent or better scientific quality and relevance for assessing risks of injury to health or the environment of chemical substances or mixtures . . . ” and to develop a strategic plan within 2 years of enactment or by June 2018 (S.697, 2016). Section 4 of the new law includes specific guidance on the use of nonanimal tests when available for initial screening and tiered testing of chemical substances and mixtures (S.697, 2016). Therefore, an accurate and internationally accepted nonanimal test for ocular irritation is needed.

In light of these issues, increased interest has focused on the development of nonanimal testing methods and strategies to replace live animal toxicity tests. Toward this end, the Interagency Coordinating Committee for the Validation of Alternative Methods (ICCVAM) and the European Centre for Validation of Alternative Methods (ECVAM) conducted retrospective evaluations of data available for four organotypic methods and four cytotoxicity and cell function test methods. Based on these retrospective evaluations, the predictive performance of all individual test methods was not felt to be sufficient for any one test, or group of tests, to fully replace the rabbit Draize eye test (ICCVAM, 2009). ICCVAM and ECVAM did, however, accept the Bovine Corneal Opacity and Permeability (BCOP) test, Isolated Chicken Eye (ICE) test, Cytosensor Microphysiometer (CM, for water-soluble materials), and Fluorescein Leakage test (for water-soluble materials) as screening tests for the identification of materials classified as NC, ocular corrosives, and severe eye irritants, and the CM as a screening test for the identification of materials classified as NC (surfactants and surfactant mixtures). Recently, differentiated cell culture models, including the EPIOCULAR™ Eye Irritation Test (EIT), the SKINETHIC™ Human Corneal Epithelium (HCE) Eye Irritation Test (EIT), and the LABCYTE CORNEA-MODEL24 Eye Irritation Test (EIT), were demonstrated to have utility for the detection of NC (OECD, 2019a).

Overall, there are a limited number of types of ocular irritation tests that do not require the use of animals. These tests include cell culture-based tests, tests based on excised animal eyes, egg-based tests, and biochemical (macromolecular) tests. All of these tests fail to identify or model some essential component of the live eye and have either poor specificity or sensitivity. The lack of understanding of the underlying reasons why some substances are much more damaging than others has hindered the development of nonanimal tests for eye safety testing. Those familiar with the state of the art say the high false-negative (FN) rates of nonanimal tests is because nonanimal tests are only able to measure initial damage, but do not accurately model the repair/reversibility of the damage.

False negatives are dangerous because the nonanimal test predicts that a chemical or product is safe for the eye, when in fact, the substance irritates or corrodes the live eye. False positives are dangerous because people do not believe test methods with a high false-positive (FP) rate resulting in ignoring warning labels, and manufacturers are slow to adopt methods with a high FP rate because they erroneously restrict the use of safe products and scare away consumers.

### SUMMARY

Disclosed are materials and methods for improving the prediction of ocular irritancy of a test substance. The method includes applying the test substance to a solid support non-biological polymer material that models the barrier function of the cellular membranes and connective tissues of the eye, measuring the migration distance, and then using the migration distance measurement to improve on an existing nonanimal tests ocular irritancy prediction; where the existing ocular irritancy test prediction can be: (1) a macromolecular test such as the OptiSafe Eye Irritation Test™ (OS EIT), (2) a cultured epithelium based test such as the EpiOcular™ Eye Irritation Test (EIT) or the Short Time Exposure (STE), (3) or and Organotypic eye irritation test such as the Bovine Corneal Opacity and Permeability (BCOP) test, Isolated Chicken Eye (ICE) test, or Hen's Egg Test-Chorioallantoic Membrane (HET-CAM) test (or similar); measuring a test system response; and multiplying or adding the measured migration distance, and predicting the ocular irritancy of the test substance based on the combined response.

In some embodiments, the material can be applied to a solid support composed of polyvinyl chloride (PVC) or a mixture of plastics composed of polyvinyl chloride (PVC) and polystyrene, in various configurations or mixtures, and a measuring tool or gradations, such that migration distance can easily be measured.

In some embodiments, the material to be tested is mixed with a colored dye to allow for easy observations of the migration distance.

In some embodiments, the first test system is a biochemical test system comprising purified or semi-purified molecules. In other embodiments, the test system comprises reconstituted human corneal epithelium (RhCE). In other embodiments, the test system is a depth of injury (DoI) test system comprising excised eyes.

In one embodiment, the first test system is selected from the OPTISAFE EYE IRRITATION TEST™ ocular irritation test, the OCULAR IRRITECTION® ocular irritation test, the EPIOCULAR™ ocular irritation test, the SKINETHIC™ ocular irritation test, the LABCYTE CORNEA-MODEL24 ocular irritation test, the MCTT HCE™ ocular irritation test, the SHORT TIME EXPOSURE (STE) ocular irritation test, the HEN'S EGG TEST—CHORIOALLANTOIC MEMBRANE (HET-CAM) ocular irritation test, the CHORIOALLANTOIC MEMBRANE (CAM) VASCULAR ASSAY (CAMVA) ocular irritation test and the DEPTH OF INJURY (DoI) ocular irritation test.

In some embodiments the established ocular irritancy classes are selected from a nonirritant, a minimal irritant, a mild irritant, and a severe irritant. In other embodiments, the established ocular irritancy classes include GHS Categories NC, 2, 2B, 2A, and 1, or EPA Categories IV, III, II, and I.

In some embodiments, the test material is polyvinyl chloride (PVC) alone or mixed bound or otherwise adhered to form a test strip.

In some embodiments, the solid platform is cardboard, cardstock paper, construction paper, plastic, glass, or similar.

A system and material to measure the biologically relevant migration distance within a tissue is disclosed. The Depth of Migration (DM) test is conducted to account for the unique penetration into the eye properties of some chemicals. This test result is multiplied by the main test score in order to adjust for the potential DoI induced by the sample being tested. The procedure involves:
a. Make the assay stock by diluting the test sample with 4.8 mL deionized (DI) water and 1.2 mL or 1.2 g of test sample in a 7-mL tube. Vortex for 10 seconds. This stock will be used for continuing the test sample dilutions representing 1%, 5%, 10%, 25%, and 50% (Table 1).

TABLE 1
Example of Test Chemical Dilution with Total Volume of 5 mL

| Dilution | Amount of | Amount of 1:10 Dilution |
|---|---|---|
| (%) | DI Water | of Test Sample |
| 1% | 5.0 mL | 0.05 mL |
| 5% | 4.75 mL | 0.25 mL |
| 10% | 4.5 mL | 0.5 mL |
| 25% | 3.75 mL | 1.25 mL |
| 50% | 2.5 mL | 2.5 mL |

DI = Deionized water; *For “1%”, 5.0 mL is used.

b. Label a 24-well plate.
c. Pipette 1.25 mL of blanking buffer (BB) to the corresponding labeled 24-well plate with a 12.5 mL Eppendorf Combitip. Pipette 1.45 mL of active agent (AA) to the corresponding labeled 24-well plate with another clean 12.5 mL Eppendorf Combitip.
d. Application of standards and quality control (QC) chemicals
   i. Vortex at maximum speed for 10 seconds the standards and quality control (QC) chemicals that come with the kit.
   ii. Add 125 μL of each standard and quality control (QC) chemicals directly into the appropriately labeled wells. Use a new pipette tip for each well to avoid cross-contamination.
   iii. Immediately after dispensing the standards or quality control (QC) chemicals, gently mix the well with the pipette tip three times. Dispose the pipette tip and repeat for the rest of the wells.
e. Application of Test Samples
   i. Vortex the test samples for 10 seconds prior to aliquoting.
   ii. Pipette 125 μL of the 1%, 5%, 10%, 25%, and 50% dilutions of each test chemical into the properly labeled BB and AA wells.
   iii. Follow the same pipette procedures for the standards and quality control (QC) chemicals.
a. Place the 24-well plate into the provided airtight incubation container and seal tightly.
b. Place the assay plate into a 30.6° C.-31.3° C. incubator for 18 h±30 min.
f. Depth of Migration (DM) Test
   i. Obtain the Depth of Migration (DM) test card. The test card should have the test strips adhered such that the migration distance markings line up with each test strip. As shown in FIG. 1A, the test card will have 5 test strips adhered to test card for the negative control, positive control, and triplicate runs of the test sample.
   ii. Obtain the Depth of Migration (DM) platform. The platform should be prepared by pulling the stand open so that the test card is sitting at a 150 angle, as shown in FIG. 1B and FIG. 1C.
   iii. Dilute the test chemical using the following dilution series:
      a. Make a 1:10 dilution of the test sample by adding 100 μL of the stock test sample to 900 μL of the provided Depth of Migration (DM) dye solution. Cap and vortex at maximum speed for 10 seconds.
      b. Using the 1:10 dilution, make the 1:100 dilution (final working stock) by adding 100 μL of the 1:10 to 900 μL of the provided Depth of Migration (DM) dye solution. Cap and vortex at maximum speed for 10 seconds.
   iv. Prior to application, vortex the negative control, positive control, and 1:100 dilution of the test article/dye solution at maximum speed for 10 seconds.
   v. Place the test card onto the platform, as shown in FIGS. 1A-1C.
   vi. Pipette 30 μL of the negative control, positive control, and 1:100 dilution of the test article/dye solution onto each of the test strips on the test card at the top of the strip (as shown in FIG. 2 and FIG. 3).
   vii. After 30-120 seconds, record the distance traveled (cm) of the leading edge (bottom of the drop; depicted by the line on FIG. 2 and FIG. 3 using the printed ruler on the test card. Input results on the data sheet.
   viii. Calculations
      i. On the data sheet, calculate the average of the triplicate test sample measurements. Subtract the negative control from the average test sample measurement to obtain the distance traveled (DT).
         1. If the distance traveled ≥1.0 cm, multiply the single highest assay score (A) by the distance traveled and a correction factor (CF) of 1.75.
         2. If the distance traveled <1.0, multiply the single highest assay score (A) by a CF of 1.0.
      ii. Using the prediction models, determine the GHS classification and the Ultra Mild and EPA predictions if needed.

In one embodiment, the material is made from polyvinyl chloride (PVC) sheets alone or with other polymers or similar that are affixed to a solid support or similar.

In one embodiment, the test chemical is diluted in the provided dye solution and placed on the migration strips, the migration distance of the drop is measured.

In one embodiment, the results of the test system are calculated by measuring the migration distance and multiplying this distance by the initial score.

In one embodiment, the results are applied to the applicable GHS or EPA classification.

In some embodiments, the formulation reduces the FN rate of in vitro nonanimal eye irritation tests, for example: Triton X-100 (10%) [CASRN 9002-93-1], an ocular corrosive chemical, without the Depth of Migration (DM) test was predicted to be a nonirritant, however, with the Depth of Migration (DM) test, this test chemical was predicted to be an ocular corrosive.

A procedure and material are disclosed for the accurate prediction of eye toxicity after a chemical, product, or material exposure in which a test chemical is assayed using two test systems to reduce the rate of mispredictions. The test chemical is applied to the Depth of Migration (DM) test system migration strips and the distance that the test chemical traveled is measured and used as a correction factor (CF) for the score of the main assay. This total score is then applied to the standardized prediction models to predict the extent of ocular toxicity, which can range from nonirritant to irritant to corrosive.

In some embodiments, irritants and corrosives may include one of more of the following: dodecanaminium, N-(2-hydroxy-3-sulfopropyl)-N,N-dimethyl-,1-naphthaleneacetic acid, 1-octanol, 1,2,4-triazole, sodium salt, 1,3-di-isopropylbenzene, 1,3-diiminobenz (f)-isoindoline, 1,5-hexadiene, 2-benzyl-4-chlorophenol, 2-benzyloxyethanol, 2-ethoxyethyl acetate (cellosolve acetate), 2-ethyl-1-hexanol, 2-hydroxyisobutyric acid ethylester, 2-hydroxyisobutyric acid, 2-methyl-1-pentanol, 2-methylbutyric acid, 2-naphthalene sulfonic acid, formaldehyde, hydroxymethylbenzene sulfonic acid monosodium salt, 2-nitro-4-thiocyanoaniline, 2,2-dimethyl-3-pentanol, 2,2-dimethyl butanoic acid, 2,5-dimethyl-2,5-hexanediol, 2,6-dichlorobenzoyl chloride, 2,6-dichloro-5-fluoro-beta-oxo-3-pyridinepropanoate, 3-chloropropionitrile, 3,3-dithiodipropionic acid, 3,4-dichlorophenyl isocyanate, 4-(1,1,3,3-tetramethylbutyl)phenol, 4-tert-butylcatechol, 4-carboxybenzaldehyde, 4-chloro-methanilic acid, 6-methyl purine, p-tert-butylphenol, acetic acid, acetone, acid blue 40, acid red 92, alpha-ketoglutaric acid alpha, ammonia, aluminum chloride, gamma-aminopropyltriethoxy silane, ammonium nitrate, antimony oxide, benzalkonium chloride, benzalkonium chloride (10%), benzenesulfonyl chloride, benzethonium chloride (10%), benzene, 1,1′-oxybis-, tetrapropylene derivatives, sulfonated, sodium salts, benzotrichloride, benzyl alcohol, beta-resorcylic acid, bis-(3-aminopropyl) tetramethyl disiloxane, butanol, butyl acetate, butyl cellosolve, butyl dipropasol solvent, butylnaphthalene sulfonic acid sodium salt, butyrolactone, calcium thioglycolate, captan 90-concentrate (solid), camphene, cetylpyridinium bromide (10%), cetylpyridinium chloride (10%), cetyltrimethylammonium bromide (10%), chlorhexidine, chloroform, cyclohexanol, cyclohexanone, cyclohexyl isocyanate, cyclopentanol, deoxycholic acid sodium salt (10%), di(2-ethylhexyl) sodium sulfosuccinate (10%), di(propylene glycol) propyl ether, dibenzoyl-L-tartaric acid, dibenzyl phosphate, diethylaminopropionitrile, domiphen bromide (10%), ethanol, ethyl 2-methyl acetoacetate, ethyl trimethyl acetate, glycidyl methacrylate, granuform, hydroxyethyl acrylate, imidazole, isobutanal, isobutyl alcohol, isopropyl alcohol, lactic acid, lauric acid, lauryldimethylamine oxide, lime, m-phenylene diamine, magnesium hydroxide, maneb, methoxyethyl acrylate, methyl acetate, methyl cyanoacetate, methyl cyclopentane, methyl ethyl ketone (2-butanone), methyl isobutyl ketone, methylpentynol, methylthioglycolate, myristyl alcohol, n-acetyl-methionine, n-butanol, n-hexanol, n-laurylsarcosine sodium salt (10%), n-octylamine, N,N,N′,N′-tetramethylhexanediamine, naphthalenesulfonic acid, 2-naphthalenesulfonic acid, sodium salt, nitric acid, organofunctional silane 45-49, phosphorodicloridic acid, hydrogenated tallow amine, polyoxyethylene(23) lauryl ether, potassium laurate (10%), potassium oleate, promethazine hydrochloride, potassium hydroxide, protectol PP, pyridine, benzyl-C12-16-alkyldimethyl, silver nitrate, sodium 2-naphthalenesulfonate, sodium hydrogen difluoride, sodium hydrogen sulfate, sodium hydroxide (10%), sodium lauryl sulfate, sodium lauryl sulfate (15%), sodium monochloroacetate, sodium oxalate, sodium perborate tetrahydrate, sodium polyoxyethylene(3) lauryl ether sulfate, sodium salicylate, stearyltrimethylammonium chloride, sulfuric acid, tetra-N-octylammonium bromide, tetraethylene glycol diacrylate, tetrahydrofuran, trichloroacetic acid (30%), trichloroacetyl chloride, triethanolamine, triethanolamine polyoxyethylene(3.0) lauryl ether sulfate, triton X-100, triton X-100 (5%), and triton X-100 (10%).

In some embodiments, known nonirritants may include one or more of the following: 1-bromo-4-chlorobutane, styrene, 1,9-decadiene, 2-ethylhexyl p-dimethylamino benzoate, 2-methylpentane, 2-(n-dodecylthio)-ethanol, 2,2-dimethyl-3-pentanol, 2,4-difluoronitrobenzene, 2,4-pentanediol, 3-methoxy-1,2-propanediol, 3-methylhexane, 3,3-dimethylpentane, acrylic acid homopolymer sodium salt, di-n-propyl disulphide, diisobutyl ketone, ethylhexyl salicylate, glycerol, iso-octyl acrylate, isopropyl bromide, isopropyl myristate, iso-octylthioglycolate, methyl trimethyl acetate, n-hexyl bromide, n-octyl bromide, n,n-dimethylguanidine sulfate, polyethylene glycol 400, polyethyleneglycol monolaurate (10 E.O.), polyoxyethylene hydrogenated castor oil (60E.O.), polyoxyethylene(14) tribenzylated phenyl ether, polyoxyethylene(160) sorbitan triisostearate, polyoxyethylene (40) hydrogenated castor oil, potassium tetrafluoroborate, propylene glycol, sodium lauryl sulfate (3%), sorbitan monolaurate, tetra-aminopyrimidine sulfate, toluene, triton X-100 (1%), and tween 80.

In some embodiments, materials to conduct the migration test include acrylonitrile butadiene styrene, allyl resin, cellulosic, epoxy, ethylene vinyl alcohol, ethylene vinyl acetate, flouroplastics, ionomer, melamine formaldehyde, phenol-formaldehyde plastic, polyacetal, polyacrylates, polyacrylonitrile, polyamide, polyamide-imide, polyaryletherketone, polybutadiene, polybutylene, polycarbonate, thermoplastic polycondensate, polydicyclopentadiene, polyektone, polyester, polyetheretherketone, thermoplastic polycondensate, polyetherimide, thermoplastic polycondensate, polyethersulfone, thermoplastic polycondensate, polyethylene, thermoplastic polymer, polyethylenechlorinates, polyimide, thermoplastic or thermoset polycondensate, polymethylpentene, polyphenylene oxide, polyphenylene sulfide, polyphthalamide, thermoplastic polycondensate, polypropylene, polystyrene, polysulfone, polyurethane, polyvinylchloride, polyvinylidene chloride, silicone, thermoset polycondensate, thermoplastic elastomers, and thermoplastic. Polysiloxane, polyphosphazene, polyborazyline, low-density polyethylene, high-density polyethylene, polypropylene, polyvinyl chloride, polystyrene, nylon, nylon 6, nylon 6,6, polytetrafluoroethylene, thermoplastic polyurethanes, polytetrafluoroethylene, polychlorotrifluoroethylene, polybutadiene, polyoxyethylene, polycarbonate, polyester, polylactic acid, polymethylmethacrylate, polyacrylonitrile, phenol-formaldehyde resin, para-aramid fiber, para-aramid, poly-paraphenylene terephthalamide, polyethylene terephthalate film, polychloroprene, polyamide, meta-aramid polymer, polyacrylonitrile, polyamide 11 & 12, copolyamid, polyimide, aromatic polyester, polyester, polytetrafluoroethylene elastomer, poly-p-phenylene-2,6-benzobisoxazole, epoxy resins, urea-formaldehyde resin, alkyd resin, polyethylene, polyisobutylene, polybutadiene, polychloroprene, acrylonitrile butadiene styrene, cross-lined polyethylene, chlorinated polyvinyl chloride, linear low-density polyethylene, thermoplastic elastomer tubing, silicone, polybutylene, high-density polybutylene, polyphenylsulfone, polysulfone, fluorinated semi-crystalline thermoplastics, polyphenylene sulfide, polythalamide, poly(cis-1,4-isoprene), poly(trans-1,4-isoprene), polyolefins, or similar.

In some embodiments, the solid support is cardboard, cardstock paper, construction paper, wood, plastic, glass, aluminum, steel or other metal, or similar.

As used herein, “toxicity” is used to refer to a substance's ability to damage, irritate, or otherwise negatively affect an eye. Toxicity may be evidenced by pain, irritation, swelling, opaqueness, redness, and discharge. Such effects may be temporary or permanent. Accordingly, the word “toxicity” is defined broadly to include any discomfort or unfavorable experience associated with the presence of a substance contacting an eye. As used herein, “irritancy” or “irritant” is used broadly to cover the spectrum of between nonirritating (nontoxic) to highly corrosive.

### BRIEF DESCRIPTION OF THE DRAWINGS

FIG. 1A-1C depicts the Depth of Migration (DM) test card, the placement of the test card onto the platform, and the measurement of the angle of the platform.

FIG. 1A depicts the Depth of Migration (DM) test card with the test strips adhered to the card and aligned with the printed ruler to the left of each test strip. There are test strips for a negative control, positive control, and three repeats of the test sample.

FIG. 1B depicts the Depth of Migration (DM) test card placed onto the Depth of Migration (DM) platform. The side view shows that the Depth of Migration (DM) test card sits flat against the platform and the angle should be 15°.

FIG. 1C depicts the measurement of the angle (15°) of the Depth of Migration (DM) platform with a protractor.

FIG. 2 depicts a completed Depth of Migration (DM) test card for a negative test sample with 30 μL aliquots of the negative control (NC), positive control (PC), and three repeats of the test sample (R1, R2, R3). The positive control aliquot traveled the entirety of the test strip.

FIG. 3 depicts a completed Depth of Migration (DM) test card for a positive test sample with 30 μL aliquots of the negative control (NC), positive control (PC), and three repeats of the test sample (R1, R2, R3). The positive control and test sample aliquots traveled the entirety of the test strip.

### DETAILED DESCRIPTION

As disclosed herein, the inventors have discovered that high false negative (FN) rates exhibited by current nonanimal ocular irritancy tests can be substantially reduced and/or prevented by accounting for material to be tested penetration into the cornea; and it was hypothesized that the penetration into the cornea depends on the physiochemical properties of membranes and connective tissues. The bases of the invention is that this penetration can be modeled using a simple non-biological polymer; and these polymers unexpectantly model the cellular membranes and connective tissues with respect to the depth of penetration into the eye variable, and the depth of penetration predicted with this simple non-biological test material can be used as a correction factor (CF) to increase the accuracy and more specifically reduce the false negative (FN) rate of a standard eye toxicity test or in some cases such as differentiated epithelium tests, expand the application of these tests to identify both eye irritation and eye damage. The high false negative (FN) rates of current nonanimal tests are likely not only because of a failure to reverse damage caused by irritants after it has occurred, but also by a failure of nonanimal tests to model other aspects of the live eye, including penetration, which theoretically increases toxicity because the chemically induced tissue and cell damage then occurs deeper into the ocular tissue, which is more sensitive and slower to repair, and more prone to scarring; and superficial damage is likely clinically scored as irritation, while deeper damage is likely clinically scored as eye damage. As described below, although biochemical, cell-based and other nonanimal eye tests have been in development for over 25 years, the depth of chemical penetration using a second non-biological test, has not been incorporated into tests or test development or strategies to reduce the false negative (FN) rate and better defined the extent of eye toxicity on a scale from none, to irritation, to eye damage. Nonetheless, as disclosed herein, are materials and methods for measuring the penetration into the eye, as well as methods for using the measured penetration as a multiplication or correction factor (CF) for the ability to damage tissues and macromolecules, reduces the FN rate and better differentiates the different classes of eye toxicity. Consequently, the specific and substantial reduction in the false negative (FN) rate as a result of using a simple non-biological test for migration distance to model penetration into the eye, is an important and unexpected finding.

Cell culture-based tests typically involve applying the test substance to epithelial tissues grown in a dish to determine its toxicity based on the degree of cells killed by the substance after a fixed time. These tests only examine the outermost layer of the eye (the epithelium). These cells do not represent the full thickness of the cornea and do not account for differences in toxicity as a function of how deep the toxin penetrates into the cornea.

Tests based on fertilized eggs, or the Hen's Egg Test-Chorioallantoic Membrane (HET-CAM), measure changes to the vessels that extend from the developing yolk to the air cell within the egg; this primitive respiratory tissue (chorioallantoic membrane, CAM) is a system that is at the very surface of the chorioallantoic membrane, and therefore, while the test measures toxicity, it does not measure the potential to penetrate into the eye.

These tests exhibit high false negative (FN) rates for the detection of materials that damage the eye and do not accurately approximate the live animal response to the same chemicals indicating they are missing a variable required reduce the false negative (FN) rate.

Although the nonanimal alternative eye toxicity tests described above have been performed for many years, toxicologists and those skilled in the art do not know all of the underlying reasons why some substances cause persistent or permanent damage to the live eye or how to correct the different in vitro models of toxicity to account for important differences between materials that cause eye irritation versus materials that cause eye damage. Nonetheless, modern toxicity classification for labeling and safety data sheets, as well as for other uses including informing users to wear eye protection, depends on accurately predicting whether the material being tested causes serious eye damage and therefore eye protection and other precautions mist be followed when using the identified as dangerous material.

### The Unique Structure of the Eye

The cornea consists of a stroma that is protected by a 5-7-layer-thick corneal epithelium. This epithelium is stratified, nonkeratinized squamous tissue. The stroma is below the epithelium and is a thicker, translucent layer composed of some cells but mostly clear connective tissue that includes a form of highly organized collagen.

The epithelium is quickly replaced (within 24 h) by sheets of cells produced by the area that encircles the cornea; the limbus (Dupps et al., 2006; Ljubimov and Saghizadeh, 2015; Torricelli et al., 2016; Mobaraki et al., 2019). On the other hand, the stroma, is slower to be replaced and if damaged or scarred can become opaque and resulting in the decrease or loss of vision. Therefore, the result of toxic exposure to the deeper stroma is significantly more severe than when just the epithelium is damaged. Nonetheless, the macromolecular and cell-based toxicity tests described above, do not account for the differences in severity related to the extent of stromal damage versus epithelial damage. The invention disclosed here can therefore be used in conjunction with toxicity tests to better account for a materials migration deep into the eye and therefore better predict the degree of severity of damage. The degree or severity of damage is directly related to the regulatory classification that is used to inform user if they need to protect their eyes when using the material to reduce the chance of permanent eye damage.

Even though scientists in the area of ophthalmology including our group, have characterized the importance of the depth of injury (DoI) (Lebrun et al., 2019; 2022), current macromolecular, cell, egg, and in vitro eye tests do not attempt to account for, and no one has figured out until now, or developed a test for the potential of a tested material to penetrate into the eye using a purely synthetic, shelf-stable, non-biological test material, to be used to combined with a toxicity test to account for the penetration and thereby obtain a more accurate prediction of the potential for eye damage. Based on our literature review, current nonanimal eye irritation tests have not specifically accounted for, and do not have a separate test to quantify migration distance or penetration into the eye, and especially do not use a synthetic non-biological, shelf stable test material to measure and then adjust the predicted effects of toxic chemicals on the eye. Current nonanimal eye irritation tests are highly simplified, reductionist models without important factors, such as accounting for migration distance in the deeper structures of the eye. Adding this variable significantly improves upon these tests by reducing the false negative (FN) rate for the detection of materials that cause eye damage.

Use of the Distance Multiplier with the Macromolecular Ocular Irritation Test

To address the need for a better and more predictive nonanimal ocular irritation test, we have been developing and improving the chemically based, in vitro ocular irritation test, referred to as OPTISAFE EYE IRRITATION TEST™. Originally, this test was developed to discriminate nonirritants from irritants/corrosives in fewer than 24 h, with only an hour of hands-on time. Furthermore, multiple test samples can be evaluated simultaneously using standard laboratory techniques and equipment with a shelf life of at least 1 year. Recently, results from a validation study showed that OPTISAFE EYE IRRITATION TEST™ provides a rapid, high-throughput screening method for eye toxins.

Since we have noted that in vitro tests, including OPTISAFE EYE IRRITATION TEST™, underpredict many of the same chemicals, we questioned whether these negatives might have common physiochemical properties.

We complied a new list that compared underpredictions for eye damage and toxicity by all the different nonanimal tests. We noticed that nonanimal tests underpredict the same chemicals that had similar chemical properties. We formulated the hypothesis that many of these false negatives were associated with the ability to penetrate deep into the eye. This is quite unexpected because the different tests have a range of properties; however, they are all have the property of being highly reductionist without considering the depth of the eye. We noticed that using a depth multiplier, generally improves the accuracy, especially by reducing the false negative (FN) rate.

### OptiSafe Eye Irritation Test™ Kit

The OptiSafe Eye Irritation Test™ Kit is available for purchase from the developer. Recently, the developer has sold 10 OptiSafe Eye Irritation Test™ kits which resulted in sales of about $18,000 (for up to 30 tests). The test kits have a shelf-life of one year and each test kit can test up to three test samples. Two boxes are shipped out (one for room temperature materials and one for frozen materials).

Box 1 (Room Temperature) components include: assay plates, dilution tubes, Blank Buffer (BB), and Depth of Migration (DM) test materials (test card, platform, dye solution, controls). Box 2 (Frozen, for storage at −20° C.) contains the Active Agent (AA) reagent.

Methods

To account for the unique properties of foaming agents, this procedure requires the Depth of Migration (DM) test. This result is multiplied by the main surfactant (SA) score in order to adjust for the depth of injury (DoI) induced by the surfactant being tested.

Make the surfactant (SA) assay stock by diluting the test sample with 4.8 mL deionized (DI) water and 1.2 mL or 1.2 g of test sample in a 7-mL tube. Vortex for 10 seconds. This stock will be used for continuing the test sample dilutions representing 1%, 5%, 10%, 25%, and 50%.

Pipette 1.25 mL of Blanking Buffer (BB) to the corresponding labeled 24-well plate with a 12.5 mL Eppendorf Combitip. Pipette 1.45 mL of Active Agent (AA) to the corresponding labeled 24-well plate with another clean 12.5 mL Eppendorf Combitip.

Application of Standards and Quality Control (QC) Chemicals
a. Vortex at maximum speed for 10 seconds the standards and quality control (QC) chemicals that come with the kit.
b. Add 125 μL of each standard and quality control (QC) chemicals directly into the appropriately labeled wells. Use a new pipette tip for each well to avoid cross-contamination.
c. Immediately after dispensing the standards or quality control (QC) chemicals, gently mix the well with the pipette tip three times. Dispose the pipette tip and repeat for the rest of the wells.

Application of test samples
a. Vortex the test samples for 10 seconds prior to aliquoting.
b. Pipette 125 μL of the 1%, 5%, 10%, 25%, and 50% dilutions of each test chemical into the properly labeled Blanking Buffer (BB) and Active Agent (AA) wells.
c. Follow the same pipette procedures for the standards and quality control (QC) chemicals.
d. Place the 24-well plate into the provided airtight incubation container and seal tightly.
e. Place the assay plate into a 30.6° C.-31.3° C. incubator for 18 h+30 min.

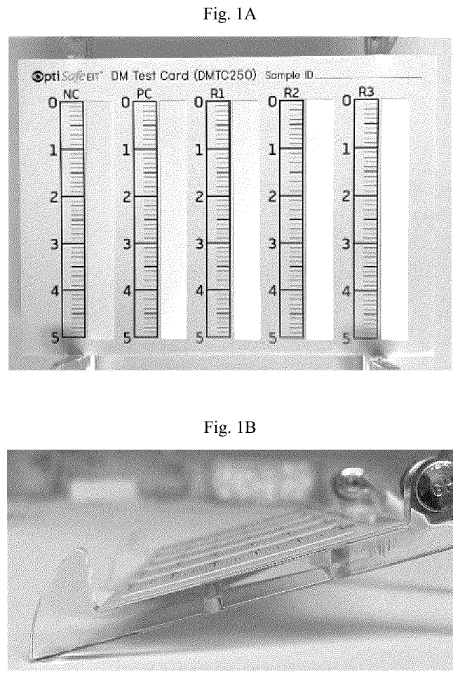

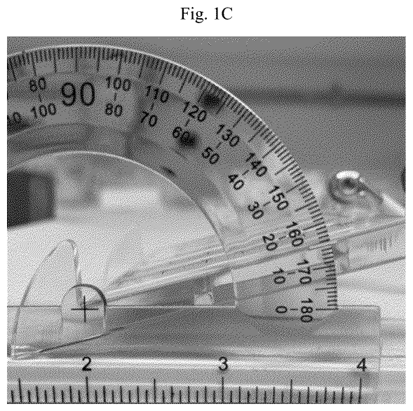

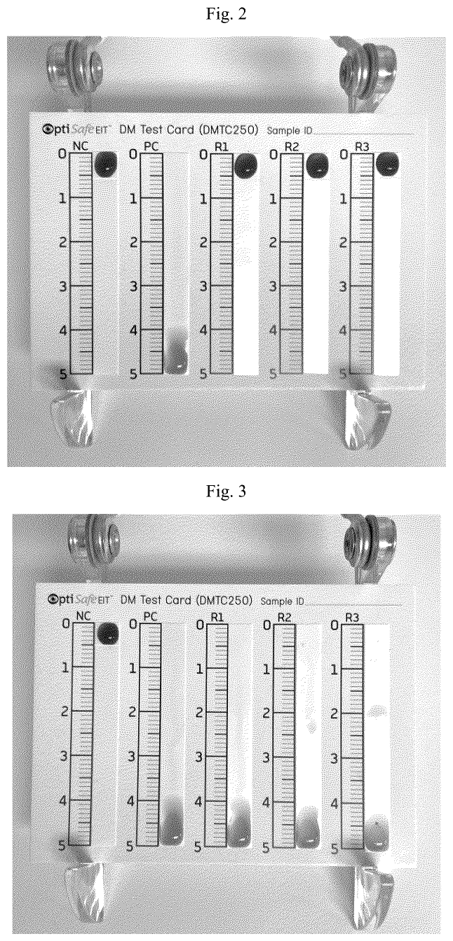

Depth of Migration (DM) Test
a. Obtain the Depth of Migration (DM) test card. The test card should have the strips adhered such that the migration distance markings line up with each strip. As shown in FIG. 1A, the test card will have 5 test strips adhered to the test card for the negative control, positive control, and triplicate runs of the test sample.
b. Obtain the Depth of Migration (DM) platform. The platform should be prepared by pulling the stand open so that the DM test card is sitting at a 15° angle, as shown in FIG. 1B and FIG. 1C.
c. Dilute the test chemical using the following dilution series:
   i. Make a 1:10 dilution of the test sample by adding 100 μL of the stock test chemical to 900 μL of the provide Depth of Migration (DM) dye solution. Cap and vortex at maximum speed for 10 seconds.
   ii. Using the 1:10 dilution, make the 1:100 dilution (final working stock) by adding 100 μL of the 1:10 to 900 μL of the provided Depth of Migration (DM) dye solution. Cap and vortex at maximum speed for 10 seconds.
d. Prior to application, vortex the negative control, positive control, and 1:100 dilution of the test article/dye solution at maximum speed for 10 seconds
e. Place the test card onto the platform, as shown in FIGS. 1A-1C.
f. Pipette 30 μL of the negative control, positive control, and 1:100 dilution of the test article/dye solution onto each of the test strips on the test card at the top of the strip (as shown in FIG. 2 and FIG. 3).
g. After 30-120 seconds, record the distance traveled (cm) of the leading edge (bottom of the drop; depicted by the line on FIG. 2 and FIG. 3) using the printed ruler on the test card. Input results on the data sheet.
h. Calculations
   i. On the data sheet, calculate the average of the triplicate test sample measurements. Subtract the negative control from the average test sample measurement to obtain the distance traveled (DT).
      1. If the distance traveled ≥1.0 cm, multiply the single highest assay score (A) by the distance traveled and a correction factor (CF) of 1.75.
      2. If the distance traveled <1.0, multiply the single highest assay score (A) by a correction factor (CF) of 1.0.
   ii. Using the prediction models, determine the GHS classification and the Ultra Mild and EPA predictions if needed.

Results

A limited set of surfactant chemicals were used to screen for the effects of ocular irritation.

The false negative (FN) rate, false positive (FP) rate, and accuracy for just the 16 surfactants was 0% (0/5), 16.7% (1/6), and 93.3% (14/15), respectively. Sodium lauroyl sarcosinate (10%) did not meet criteria (CNM) due to assay inhibition (see Choksi et al., 2020).

Triton X-100 (10%) [CASRN 9002-93-1] was mispredicted before the Depth of Migration (DM) test. The Depth of Migration (DM) test correctly predicted the test chemical.

Considering all results, including the Depth of Migration (DM) test, for the prediction of irritation, the false negative (FN) rate was 0% (0/89), the false positive (FP) rate was 20.4% (10/49), and the accuracy was 92.8% (128/138).

Based on the reduction of the false negative (FN) rate to zero, we concluded that the consideration of distance migrated to nonanimal tests is critical to lower the negative rate for ocular toxins that damage the eye. This improvement is unexpected, and while nonanimal tests for eye safety have been done for 25 years, only now has the importance of this been recognized. The consideration of migration distance appears to be required for the accurate and specific modeling of eye safety after chemical or product exposure. Measuring this on a synthetic test material and then using the measured value to correct another toxicity test has never before been described with respect to in vitro, nonanimal test methods.

### Material Compositions

In certain embodiments of the various in vitro ocular irritation test methods, a Depth of Migration (DM) multiplier can be employed to improve the prediction of toxicity to the eye. That material may utilize any combination of polypropylene, polystyrene, low-density, acrylate, polyethylene, high-density polyethylene, polyvinyl chloride, ethylene vinyl acetate, polyesters, polyurethanes, polyolefins, or similar.

In some embodiments, the material is acylate, polyvinyl chloride, ethylene vinyl acetate, polypropylene, polystyrene, or similar.

The test will preferably also include a dye to allow for easy measurement of migration. The dye can be: Brilliant Blue G, Brilliant Blue R, Brilliant Blue FCF, Brilliant Cresyl Blue ALD, Ponceau S, Bromophenol Blue, Brilliant Green, Orange II Sodium Salt, Fluorescein Sodium Salt, Evans Blue, Methylene Blue, Toluidine Blue O, Trypan Blue, Alcian Blue 8GX, Indigo, Indigo Carmine, Methyl Blue, Fast Blue B Salt, Solvent Green 3, Lissamine Green B, Bromocresol Green, Acid Green 25, Janus Green B, Methyl Green, Fast Green FCF, Quinaldine Red, Congo Red, Direct Red 80, Amaranth, Para Red, Direct Red 81, Disperse Red 1, Methyl Red, Cresol Red, Fast Red Violet LB Salt, Neutral Red, Phenol Red Sodium Salt, Orange G, Disperse Orange 37, Methyl Orange, Bismarck Brown Y, Auramine O, Carbol-Fuchsin, Resazurin Sodium Salt, Lucifer Yellow, Sulforhodamine B Sodium Salt, etc.

Other test components may include the solid support, migration strips, tubes, rulers, deionized water, dye solution, directional inserts, etc. as required to make a complete test kit.

### Experimental Details and Examples

Distance Migrated used to Improve the Accuracy of a Biochemical Test

The Depth of Migration (DM) test improves the predictive capacity of a biochemical test for eye damage. The procedure was:
1) Obtain the Depth of Migration (DM) test card. The test card should have the test strips adhered such that the migration distance markings line up with each test strip. As shown in FIG. 1A, the test card will have 5 test strips adhered to test the negative control, positive control, and triplicate runs of the test sample.
2) Obtain the Depth of Migration (DM) platform. The platform should be prepared by pulling the stand open so that the test card is sitting at a 150 angle, as shown in FIG. 1B and FIG. 1C.
3) Dilute the test chemical using the following dilution series:
   a. Make a 1:10 dilution of the test sample by adding 100 μL of the stock test sample to 900 μL of the provided Depth of Migration (DM) dye solution. Cap and vortex at maximum speed for 10 seconds.
   b. Using the 1:10 dilution, make the 1:100 dilution (final working stock) by adding 100 μL of the 1:10 to 900 μL of the provided Depth of Migration (DM) dye solution. Cap and vortex at maximum speed for 10 seconds.
4) Prior to application, vortex the negative control, positive control, and 1:100 dilution of the test article/dye solution at maximum speed for 10 seconds.
5) Place the test card onto the platform, as shown in FIGS. 1A-1C.
6) Pipette 30 μL of the negative control, positive control, and 1:100 dilution of the test article/dye solution onto each of the test strips on the test card at the top of the strip (as shown in FIG. 2 and FIG. 2).
7) After 30-120 seconds, record the distance traveled (cm) of the leading edge (bottom of the drop; depicted by the yellow line on FIG. 2 and FIG. 3 using the printed ruler on the test card. Input results on the data sheet.
8) Calculations
   a. On the data sheet, calculate the average of the triplicate test sample measurements. Subtract the negative control from the average test sample measurement to obtain the distance traveled (DT).
      i. If the distance traveled ≥1.0 cm, multiply the single highest assay score (A) by the distance traveled and a correction factor (CF) of 1.75.
      ii. If the distance traveled <1.0, multiply the single highest assay score (A) by a CF of 1.0.
b. Using the prediction models, determine the GHS classification and the Ultra Mild and EPA predictions if needed.

Pretest and Surfactant Check
1) Make “10% dilution” of substance in screw-cap glass test tubes in 2 mL OptiSafe Eye Irritation Test™ Blanking Buffer (BB) by adding 200 μL/200 mg test sample into a screw-cap glass tube.
2) Add 2 mL of the pH-adjusted Blanking Buffer (BB) to labeled tube [for colored test samples, adjust the pH of the Blanking Buffer (BB) to 6.36 using dilute (0.1 N, etc.) NaOH or dilute (0.1 N, etc.) HCl].
3) Cap tube and invert three times. Vortex mix at 45-degree angle at maximum speed for 10 sec.
4) Place tubes in a rack on bench top and allow the tube to sit undisturbed for 5-10 min.
5) Inspect tube; pick up and hold to light. If all of the substance is at the meniscus and blocks the observation, repeat the procedure at a 1% dilution if needed. Measure from the meniscus up using a metric ruler. If froth extends greater than 0.2 cm above the meniscus and bubbles are present (in either tube), the substance is classified as a “surfactant” using this method.
6) Inspect the 10% tube from the sides and bottom. If the substance is a liquid and has not mixed with the OptiSafe Eye Irritation Test™ Blanking Buffer (BB), starts to form “oily” droplets at the top or middle or bottom, or is unclear whether the substance is mixed (all clear liquids), conduct the A procedure (in addition to the a procedure).

Completely Insoluble Check
1) If the substance is a solid, let the 10% solution (made above) sit undisturbed for 30 min (up to 4 h). Note if the substance is a solid and the majority floats to the top (“F”), aggregates in the middle (“A”), or sinks (“S”) to the bottom of the tube; note this and remove approximately 0.5 mL of the liquid portion (and avoid solid portion) using a 1-mL serological pipette. Record this in procedure notes section.
2) Set the spectrophotometer to 400 nm and blank.
3) Measure the OD₄₀₀ of the recovered solution.
4) If the substance is a solid and the measured value is less than 0.350, follow the 42-h completely insoluble protocol. See Section Ci if the substance floats/aggregates/clumps or sinks.
5) If the OD is greater than 0.350, the substance is not completely insoluble.

H-Buffering Score Pretest
1) Measure the buffering power of an unknown and calculate the H-buffering score by adding a 125 μL or, if solid, a 125 mg (±10%) aliquot of test sample into a 7-mL tube.
2) Select the 8 mL tube of frozen Active Agent (AA). Use a 10-mL beaker with “mini” stir bar. Prepare the small vial of the pH-adjust solution by adding 2.5 mL deionized water and invert/shake 5-8 times. Optional: Dilute the pH-adjust solution 1:3 and/or 1:10 in deionized water (if needed for fine pH adjustments).
3) Record the starting pH of the Active Agent (AA) (6.36) on the pretest data sheet.
4) Add 1.25 mL of pH-adjusted Active Agent (AA) into a plastic 7-mL tube.
5) Cap tube and vortex mix for 5 sec at maximum speed.
6) Place tube on bench top and allow it to sit for 5 min 30 sec. In cases where the solution pH does not stabilize, continue incubation until the pH stabilizes; record this pH value and note the time until pH stability reached.
7) Measure and record the final pH on the pretest data sheet.
8) Subtract the final pH from the starting pH. Use this absolute value as the exponent (base
10). Add a subscript A or B to indicate acidic or basic buffering. The resulting value is the H-Buffering Score.
9) If the resulting H-Buffering Score is: Less than 5.0A or 2.0B, the substance is within the MA or Ci (floats/aggregates or sinks) application domains; Not MA but less than 100A or 100B, the substance is within the *HMA application domain; Greater than 100A or B, then follow the H procedure. (*Note: In the event a test substance is both completely insoluble and the H score is less than 100.0 but greater than 5.0A or 2.0B, use best scientific judgment to select one or the other.)

Alpha Procedure
1) Pre-weigh solid test samples and place membrane discs before starting.
2) Adjust the pH of the Blanking Buffer (BB) to 6.36 using NaOH. (Only required for colored test samples.)
3) Remove the 40-mL tube of frozen Active Agent (AA) solution from the freezer. Record the lot number and then warm the Active Agent (AA) solution in a 26° C.-28° C. water bath. The water level should be the same height as the Active Agent (AA) within the tube. The Active Agent (AA) should sit in the water bath for 40 min (50 min maximum).
4) Transfer the Active Agent (AA) to a 50-mL beaker with a stir bar. Allow good vortexing but minimal foaming. Add the antioxidant mixture at a specified concentration. Calibrate the pH meter and confirm its functionality by comparing measurements with a second calibrated pH meter. Record the initial pH. Reconstitute the larger aliquot of the pH-adjust solution. Add 10 mL of deionized water and screw the cap tight. Shake/invert 5-8 times. Use immediately; this mixture is single use only.
5) Slowly add the specified amount of antioxidant mixture to the Active Agent (AA) using a stir bar and magnetic plate to incorporate. Allow mixing until completely solubilized. Note any changes for quality assurance records. (Optional: Pre-add the antioxidant mixture to the Active Agent (AA) before freezing the initial lot of Active Agent (AA). Follow step 5 to incorporate into reagent.)
6) Adjust the pH to exactly 6.36 using the reconstituted pH-adjust solution: add dropwise and do not overshoot. When close to the desired pH value, allow at least 10 sec between drops to ensure equilibrium is reached. A pH adjustment will typically take 5-10 min and should not exceed 15 min.
7) Label the provided 24-well plates (when multiple tests of the same type are performed at the same time, only one set of standards plate is used).
8) Pipette 1.25 mL Blanking Buffer (BB) solution into the corresponding labeled 24-well-plates with a 12.5-mL Eppendorf Combitips (or equivalent).
9) Pipette 1.25 mL of pH-adjusted (6.36) Active Agent (AA) solution into the corresponding labeled 24-well plates with another 12.5-mL Eppendorf Combitips.
10) If the substance is a solid with a pretest solubility check optical density (OD) at 400 nm greater than 0.350, or a liquid, place membrane discs into each well (if solid, this should already have substance within it). Carefully lift the 24-well plates and check the bottom to make sure there are no bubbles between the contacting membrane and solutions. Gently tap the side of the plate or lift the discs to get rid of any bubbles.
11) Label each plate on the top and side using a Sharpie pen. Optional: Label the lid of each 24-well plate as indicated above and place the respective lid above the 24-well plates to be used as a guide. For liquid samples, doses of 25 μL, 50 μL, 75 μL, 100 μL, and 125 μL will be used. For solid samples, doses of 50 mg, 100 mg, 150 mg, 200 mg, and 250 mg will be used.
12) Pipette 125-μL triplicates of Standard 0 into three wells: triplicate Standard IV, triplicate of Standard III, and singles of Quality Control 1 and Quality Control 2 into the appropriate wells.
13) Pipette 25 μL, 50 μL, 75 μL, 100 μL, and 125 μL of the unknown test sample into the appropriate Blanking Buffer (BB) and Active Agent (AA) wells.
14) Pipette 125 μL (or 250 mg if solid, pre-weighed) of the test sample into the wells labeled TN+ and TNB.
15) Add 125 μL TN for liquid samples or 250 μL TN for solid samples to the three wells on the left side labeled TN+, TN−, and TNB (directly to center of well). If solid, mix substance with pipette tip to ensure at least some of the solubilized test substance is in contact with the membrane at the bottom of the sample well.
16) Place each 24-well plate in container provided. Seal the container by pressing firmly on all edges and then along the entire edge of box. Replace damaged incubation boxes that do not form good seal.
17) Place the entire container in a 30.6° C.-31.3° C. incubator and set a timer for 18 h. Incubation time should be 18-19 h (optimum 18 h, time is allowed when conducting multiple assays at the same time).

Reading the 400 nm (a) Assay Results
1) Turn on the spectrophotometer for at least 10 min before use.
2) Adjust the spectrophotometer to 400 nm, absorbance mode. Press “esc,” Press “General Tests,” Press “Basic ATC,” Press “Set Wavelength,” enter “400,” and then press “enter.” If not in absorbance mode, press “mode.” Adjust to absorbance mode. Readout should include “A” at the end.
3) Blank the spectrophotometer: Add 1 mL Blanking Buffer (BB) solution to a cuvette, place in the spectrophotometer, and push the blank button. The spectrophotometer should read “0.000A” (+/−0.002).
4) After the assay incubation time is complete, remove the plate from the incubator; remove the ocular discs from each well and dispose of them appropriately. Check each disc for damage. Make a record of damaged discs or any other abnormality (precipitate, color, etc.). Fluid in the disc is normal; it is related to denaturation via osmotic forces.
5) Mix the solution in each well individually with the tip of a 1-mL pipette prior to measuring the optical density (OD). This involves rapidly and forcefully scraping the bottom of the well with a standard 1-mL pipette tip five times in one direction (in rapid zig-zag pattern), scraping the tip around the bottom edge 2-3 times, and then repeating two more times (for a total of three) to solubilize any white precipitate that has formed on the bottom of the well of the 24-well plate. Mix with force and attempt to hear an audible scraping sound (not possible with some viscous fluid samples). The precipitate at the bottom may not be visible. The precipitate at the bottom does not readily go into solution. Forceful and complete mixing is required for accurate results. After mixing, immediately aspirate the solubilized solution from the center bottom of the well.
6) Immediately aspirate 500 μL into a test cuvette after mixing. Tap the cuvette on the bench top to allow any bubbles to rise to the surface.
7) Immediately measure the optical density (OD) at 400 nm using a recently blanked (with Blanking Buffer [BB]) spectrophotometer.
8) Include negative values on the data capture sheet.
9) Record the result and any notes on the data capture sheet. (If readings fail to stabilize, consult the supplier for additional procedural information).

Depth of Migration (DM) Test
1) Obtain the Depth of Migration (DM) test card. The test card should have the test strips adhered such that the migration distance markings line up with each test strip. As shown in FIG. 1A, the test card will have 5 test strips adhered to test the negative control, positive control, and triplicate runs of the test sample.
2) Obtain the Depth of Migration (DM) platform. The platform should be prepared by pulling the stand open so that the test card is sitting at a 15° angle, as shown in FIG. 1B and FIG. 1C.
3) Dilute the test chemical using the following dilution series:
   a. Make a 1:10 dilution of the test sample by adding 100 μL of the stock test sample to 900 μL of the provided Depth of Migration (DM) dye solution. Cap and vortex at maximum speed for 10 seconds.
   b. Using the 1:10 dilution, make the 1:100 dilution (final working stock) by adding 100 μL of the 1:10 to 900 μL of the provided Depth of Migration (DM) dye solution. Cap and vortex at maximum speed for 10 seconds.
4) Prior to application, vortex the negative control, positive control, and 1:100 dilution of the test article/dye solution at maximum speed for 10 seconds.
5) Place the test card onto the platform, as shown in FIGS. 1A-1C.
6) Pipette 30 μL of the negative control, positive control, and 1:100 dilution of the test article/dye solution onto each of the test strips on the test card at the top of the strip (as shown in FIG. 2 and FIG. 2).
7) After 30-120 seconds, record the distance traveled (cm) of the leading edge (bottom of the drop; depicted by the yellow line on FIG. 2 and FIG. 3 using the printed ruler on the test card. Input results on the data sheet.
8) Calculations
   a. On the data sheet, calculate the average of the triplicate test sample measurements. Subtract the negative control from the average test sample measurement to obtain the distance traveled (DT).
      i. If the distance traveled ≥1.0 cm, multiply the single highest assay score (A) by the distance traveled and a correction factor (CF) of 1.75.
      ii. If the distance traveled <1.0, multiply the single highest assay score (A) by a CF of 1.0.
   b. Using the prediction models, determine the GHS classification and the Ultra Mild and EPA predictions if needed.

Data Analysis
1) Sample OD values are recorded under the column “Sample OD” for each respective concentration.
2) Blank OD values are recorded under the column “Blank OD” for each respective concentration.
3) Measured values (MV=Net OD values) are obtained by subtracting the Blank OD value and the average standard 0 OD value from the Sample OD values for each respective concentration. (Note: If less than zero, enter 0.)
4) A standard value is obtained by subtracting the average standard 0 value from the average of the MVs for the standards.
5) For each measured unknown test value (MV), assign a numerical value (“score”) by using the closest standard value (CSV).
6) Identify the standard with the closest OD value (either standard IV or III). Divide the measured OD by the closest standard value (MV/CSV) and multiply this value by the CSV designation (DV) (either IV=8.0 or III=12.5).
7) The resulting value=irritation score for the sample in question. Populate the template with both OD values and irritation scores.
8) Calculate the TN Value: Subtract both the TNB OD and the TN− OD from the TN+ OD.
9) Calculate the irritant score for the TN following steps above. If the TN score is the highest score, base the irritancy prediction on the TN score using the prediction models. Disregard negative TN MVs and scores.
10) For the assay results (25-125 titrations) and the TN, convert the calculated irritation scores into EPA and GHS categories using the prediction models. Only the highest score (including TN) is used for the final prediction.
11) Depth of Migration (DM) Calculations
   a. On the data sheet, calculate the average of the triplicate test sample measurements. Subtract the negative control from the average test sample measurement to obtain the distance traveled (DT).
      i) If the distance traveled ≥1.0 cm, multiply the single highest assay score (A) by the distance traveled and a correction factor (CF) of 1.75.
      ii) If the distance traveled <1.0, multiply the single highest assay score (A) by a CF of 1.0.

The false negative (FN) rate was significantly less when the distance migrated test was also used. The reduction of the false negative (FN) rate is attributed to the multiplication of the main test value by the distance migrated value.

### Additions to Tissue Culture Media for EPIOCULAR™, Short Time Exposure (STE), and Other Cell- or Tissue-Based Tests for Ocular Toxicity

To include the antioxidant mixture in cell-based assays, the mix can be added to the medium or the test sample. Per the EPIOCULAR™ protocol by MatTek Corporation, the EPIOCULAR™ Assay Medium should be warmed to approximately 37° C. The antioxidant mixture, containing one or more of the following: GSH, L-cysteine, L-tyrosine, ascorbic acid, and/or uric acid, is added to and solubilized in the medium until a homogenous mixture is reached. The tested range of ascorbic acid concentration included in the EPIOCULAR™ test method was from 1,703.4 to 17,033.8 μM. Following this, 1.0 mL of assay medium is aliquoted into the appropriate wells of pre-labeled 6-well plates, the tissues should be removed from the 24-well plates, and the insert is then transferred into the 6-well plates and preincubated in the assay medium (MatTek Corporation, 2021). Similarly, the antioxidant mixture can be applied to the media for the STE test method (ICCVAM-NICEA™, 2013).

Modified EPIOCULAR™ The following procedures were adapted from EPIOCULAR™ Eye Irritation Test (OCL-200-EIT) (2021) by MatTek Corporation (Available at: https: www.mattek.com wp-content uploads OCL-200-EIT-Eye-Irritation-Test-Protocol-MK-24-007-0055_02_02_2021.pdf). Some procedures are from the MatTek EpiOcular™ Eye Irritation Test (OCL-200-EIT) Protocol. Additional new steps to the procedure are included as steps 2, 14, 15, and 17-19.

Tissue Preincubation
1) RhCE tissues (purchased from MatTek Corporation) are equilibrated for about 15 min.
2) Cell culture medium was warmed to approximately 37° C. and one mL aliquoted into the appropriate wells of 6-well plates.
3) Tissues were removed from the shipping containers forceps and then into the 6-well plates and incubated at standard culture conditions for 1 h, then assayed medium changed and incubated overnight (16-24 h).

Test Substance Exposure
1) Test Article Exposure: After the 30±2-min pretreatment, each tissue is tested by applying 50 μL of substance to be tested topically. The tissues were then incubated at standard culture conditions for 30±2 min.
2) Dose procedure: The test article used was styrene (CASRN 100-42-5, Lot No. MKCM4502). 50 μL of styrene was added to the tissue insert.
3) Rinsing: At the end of the 30-min test chemical exposure time, the test articles are removed by rinsing the tissues.

Modifications Required for Solids
1) Each solid is tested by applying one leveled volumetric spoonful of material to be tested into tissue insert (approximately 50 mg). and incubated for 6 h.
2) Rinsing: At the end of the 6 h, the test articles are removed by rinsing (as above).
3) Post-treatment: After rinsing, the tissue inserts are immersed in 5 mL of previously warmed assay medium
4) Recovery: Next, each insert is removed from the assay medium, and transferred to a plate containing 1 mL of medium. The tissues are then incubated for 18 h under cell culture. Cell Viability Test
1) After the post-treatment incubation, the MTT viability test is done.
2) A 1.0 mg/mL MTT solution is prepared and 300 μL of the MTT solution is added to each well of a 24-well plate. each insert is removed from the 6-well plate and placed into the 24-well plate containing 0.3 mL of MTT solution. The plate is then incubated for 3 h under culture conditions.
3) After incubation, each insert is removed from the 24-well plate and then transferred to a 24-well plate containing 2.0 mL of isopropanol. The plates are sealed with saran wrap and are either stored overnight with refrigeration. After this, tissue inserts are pierced and the liquid is decanted into the well from which it came.
4) The extract solution is mixed and two 0.2 mL aliquots from each well transferred into a 96-well plate.
5) The absorbance at 570 nm (OD₅₇₀) of each well is measured with a plate reader. If the MTT OD570 is 60% or greater of that generated by the negative control, the material tested is classified as a nonirritant. On the other hand, if the test substance OD₅₇₀ is less than 60% of the negative control, the test material is considered an ocular irritant.

### Prophetic Example

EpiOcular™ false negatives include: 1,4-dibutoxy benzene, methyl cyanoacetate, 2-pseudoionone, iminodibenzyl, and bifenthrin. To perform the Depth of Migration (DM) test, take these test samples and make 1:100 working stock dilutions. Obtain Depth of Migration (DM) test cards and apply 30 μL of the negative control, positive control, and the triplicates of the test samples. Measure the distance traveled. These test samples all had a distanced traveled of 5.0 cm and are therefore the main assay results are multiplied by the distance traveled and correction factor of 1.75 (Table 3). With the Depth of Migration (DM) multiplier, the false negatives are predicted to be Category 1. The false negative (FN) rate was significantly less when the distance migrated test was accounted for. The reduction of the false negative (FN) rate is attributed to the multiplication of the main test value by the depth of migration correction factor.

TABLE 2
EpiOcular ™ Prediction Model

| Viability | EpiOcular Classification | GHS Classification |
|---|---|---|
| ≤60% | Irritant | Category 2 or greater |
| >60% | Nonirritant | No Category |

TABLE 3
Depth of Migration (DM) Prophetic Results

| | | | DM | DM |
|---|---|---|---|---|
| | | in vivo | Distance | Correction |
| Chemical Name | CASRN | GHS | Traveled | Factor |
| 1,4-dibutoxy | 104-36-9 | Cat. 2 | 4.5 | 1.75 |
| benzene | | | | |
| Methyl | 105-34-0 | Cat. 2 | 4.5 | 1.75 |
| cyanoacetate | | | | |
| 2-Pseudoionone | 141-10-6 | Cat. 2 | 4.5 | 1.75 |
| Iminodibenzyl | 494-19-9 | Cat. 1 | 4.5 | 1.75 |
| Bifenthrin | 82657-04-3 | Cat. 2 | 4.5 | 1.75 |

CASRN = Chemical Abstracts Service Registry Number; GHS = Globally Harmonized System of Classification and Labeling of Chemicals; Cat. = Category; DM = Depth of Migration.

SHORT TIME EXPOSURE (STE) The procedures are available from the NICEA™ Review Document (2013) short Time Exposure (STE) Test Method Summary Review Document (Available at: https: ntp.niehs.nih.gov iccvam docs ocutox_docs ste-srd-niceatm-508.pdf). Additional steps are included as steps 1 and 2.

### Prophetic Example

Short Time Exposure (STE) false negatives include: methyl cyanoacetate, 2,5-dimethyl-2,5-hexanediol, myristyl alcohol, propasol solvent P, potassium sorbate, 1-(1-methyl-2-propoxyethoxy)propan-2-ol, 2,6-dichlorobenzoyl chloride, sodium salicylate, ethanol, ammonium nitrate, isopropanol, methyl acetate, camphene, and hydroxyethyl acrylate. To perform the Depth of Migration (DM) test, take these test samples and make 1:100 working stock dilutions. Obtain Depth of Migration (DM) test cards and apply 30 μL of the negative control, positive control, and the triplicates of the test samples. Measure the distance traveled. These test samples all had a distanced traveled of 5.0 cm and are therefore predicted to be Category 1. The false negative (FN) rate was significantly less when the distance migrated test was accounted for. The reduction ofthe false negative (FN) rate is attributed to the multiplication of the main test value by the depth of migration correction factor.

TABLE 4
Short Time Exposure (STE) Prediction Model

Cell Viability

| At 5% | At 0.05% | GHS Classification |
|---|---|---|
| >70% | >70% | No Category |
| ≤70% | >70% | Category 2 |
| ≤70% | ≤70% | Category 1 |

TABLE 5
Depth of Migration (DM) Prophetic Results

| | | | DM | DM |
|---|---|---|---|---|
| | | in vivo | Distance | Correction |
| Chemical Name | CASRN | GHS | Traveled | Factor |
| Methyl cyanoacetate | 105-34-0 | Cat. 2 | 4.5 | 1.75 |
| 2,5-Dimethyl-2,5- | 110-03-2 | Cat. 1 | 4.5 | 1.75 |
| hexanediol | | | | |
| Myristyl alcohol | 112-72-1 | Cat. 2 | 4.5 | 1.75 |
| Propasol solvent P | 1569-01-3 | Cat. 2 | 4.5 | 1.75 |
| Potassium sorbate | 24634-61-5 | Cat. 2 | 4.5 | 1.75 |
| 1-(1-methyl-2- | 29911-27-1 | Cat. 2 | 4.5 | 1.75 |
| propoxyethoxy)propan- | | | | |
| 2-ol | | | | |
| 2,6-Dichlorobenzoyl | 4659-45-4 | Cat. 2 | 4.5 | 1.75 |
| chloride | | | | |
| Sodium salicylate | 54-21-7 | Cat. 1 | 4.5 | 1.75 |
| Ethanol | 64-17-5 | Cat. 2 | 4.5 | 1.75 |
| Ammonium nitrate | 6484-52-2 | Cat. 2 | 4.5 | 1.75 |
| Isopropanol | 67-63-0 | Cat. 2 | 4.5 | 1.75 |
| Methyl acetate | 79-20-9 | Cat. 2 | 4.5 | 1.75 |
| Camphene | 79-92-5 | Cat. 2 | 4.5 | 1.75 |
| Hydroxyethyl acrylate | 818-61-1 | Cat. 1 | 4.5 | 1.75 |

CASRN = Chemical Abstracts Service Registry Number; GHS = Globally Harmonized System of Classification and Labeling of Chemicals; Cat. = Category; DM = Depth of Migration.

### Additions to Bovine Corneal Opacity and Permeability (BCOP), Isolated Chicken Eye (ICE), Isolated Rabbit Eye (IRE), and any Other Ex Vivo Eye Test System

1) Use of the distance migration correction for ex vivo assays such as Bovine Corneal Opacity and Permeability (BCOP), Isolated Chicken Eye (ICE), Isolated Rabbit Eye (IRE), etc. can improve the prediction. Bovine Corneal Opacity and Permeability (BCOP) false negatives include: 4-(1,1,3,3-tetramethylbutyl)phenol, quinacrine, L-aspartic acid, (2R,3R)-3-((R)-1-(tert-butyldimethylsiloxy)ethyl)-4-oxoazetidin-2-yl acetate, camphene, and ethyl 2,6-dichloro-5-fluoro-beta-oxo-3-pyridine propionate. To perform the Depth of Migration (DM) test, take these test samples and make 1:100 working stock dilutions. Obtain Depth of Migration (DM) test cards and apply 30 μL of the negative control, positive control, and the triplicates of the test samples. Measure the distance traveled. These test samples all had a distanced traveled of 5.0 cm and are therefore predicted to be Category 1. The false negative (FN) rate was significantly less when the distance migrated test was accounted for. The reduction of the false negative (FN) rate is attributed to the multiplication of the main test value by the depth of migration correction factor.

TABLE 6
Bovine Corneal Opacity and Permeability
(BCOP) Prediction Model

| IVIS | GHS Classification |
|---|---|
| ≤3 | No Category |
| 3 < IVIS ≤ 55 | Category 2 |
| IVIS > 55 | Category 1 |

TABLE 7
Depth of Migration (DM) Prophetic Results

| | | in | DM | DM |
|---|---|---|---|---|
| | | vivo | Distance | Correction |
| Chemical Name | CASRN | GHS | Traveled | Factor |
| 4-(1,1,3,3- | 140-66-9 | Cat. 1 | 4.5 | 1.75 |
| Tetramethylbutyl)phenol | | | | |
| Quinacrine | 69-05-6 | Cat. 1 | 4.5 | 1.75 |
| L-Aspartic acid | 70-47-3 | Cat. 2 | 4.5 | 1.75 |
| (2R,3R)-3-((R)-1-(tert- | 76855- | Cat. 2 | 4.5 | 1.75 |
| Butyldimethylsiloxy)ethyl)- | 69-1 | | | |
| 4-oxoazetidin-2-yl acetate | | | | |
| Camphene | 79-92-5 | Cat. 2 | 4.5 | 1.75 |
| Ethyl 2,6-dichloro-5- | 96568- | Cat. 2 | 4.5 | 1.75 |
| fluoro-beta-oxo-3-pyridine | 04-6 | | | |
| propionate | | | | |

CASRN = Chemical Abstracts Service Registry Number; GHS = Globally Harmonized System of Classification and Labeling of Chemicals; Cat. = Category; DM = Depth of Migration.

2) Isolated Chicken Eye (ICE) false negatives include: methyl cyanoacetate, maneb, and 4-carboxybenzaldehyde, Any surfactant identified chemicals should be assayed using the DM test. If the dose travels more than 1.0 cm, multiply the distance traveled by the a score and the CF of 1.75 to obtain the final score. The false negative (FN) rate was significantly less when the distance migrated test was also used. The reduction of the false negative (FN) rate is attributed to the multiplication of the main test value by the distance migrated value. To perform the Depth of Migration (DM) test, take these test samples and make 1:100 working stock dilutions. Obtain Depth of Migration (DM) test cards and apply 30 μL of the negative control, positive control, and the triplicates of the test samples. Measure the distance traveled. These test samples all had a distanced traveled of 5.0 cm and are therefore predicted to be Category 1. The false negative (FN) rate was significantly less when the distance migrated test was accounted for. The reduction of the false negative (FN) rate is attributed to the multiplication of the main test value by the depth of migration correction factor.

TABLE 8
Isolated Chicken Eye (ICE) Prediction

| | GHS |
|---|---|
| Combination of 3 Endpoints | Classification |
| 3 × I | No Category |
| 2 × I, 1 × II | |
| 2 × II, 1 × I | |
| Other combinations | Category 2 |
| 3 × IV | |
| 2 × IV, 1 × III | |
| 2 × IV, 1 × II* | |
| 2 × IV, 1 × I* | Category 1 |
| Corneal opacity = 3 at 30 min (in at least 2 eyes) | |
| Corneal opacity = 4 at any time point (in at least 2 eyes) | |
| Severe loosening of the epithelium (in at least 1 eye) | |

TABLE 9
Depth of Migration (DM) Prophetic Results

| | | | DM | DM |
|---|---|---|---|---|
| | | in vivo | Distance | Correction |
| Chemical Name | CASRN | GHS | Traveled | Factor |
| Methyl cyanoacetate | 105-34-0 | Cat. 2 | 4.5 | 1.75 |
| Maneb | 12427-38-2 | Cat. 2 | 4.5 | 1.75 |
| 4-carboxybenzaldehyde | 619-66-9 | Cat. 2 | 4.5 | 1.75 |

CASRN = Chemical Abstracts Service Registry Number; GHS = Globally Harmonized System of Classification and Labeling of Chemicals; Cat. = Category; DM = Depth of Migration.

### Additions to Hen's Egg Test-Chorioallantoic Membrane (HET-CAM) or Chorioallantoic Membrane (CAM) Vascular Assay (CAMVA)

The Hen's Egg Test-Chorioallantoic Membrane (HET-CAM) procedure is described by the ICCVAM-Recommended Test Method Protocol: Hen's Egg Test Chorioallantoic Membrane (HET-CAM) Test Method (2010) (available at: https: ntp.niehs.nih.gov iccvam docs/protocols ivocular-hetcam.pdf).

Any surfactant identified chemicals should be assayed using the Depth of Migration (DM) test. Obtain Depth of Migration (DM) test cards and apply 30 μL of the negative control, positive control, and the triplicates of the test samples. Measure the distance traveled. These test samples all had a distanced traveled of 5.0 cm and are therefore predicted to be Category 1. The false negative (FN) rate was significantly less when the distance migrated test was accounted for. The reduction of the false negative (FN) rate is attributed to the multiplication of the main test value by the depth of migration correction factor.

### Additions to Acute Toxicity and any Other Digestive Irritation Test Systems

The Acute Oral toxicity test method procedure is described by the Organization for Economic Cooperation and Development (OECD) Series on Testing and Assessment No. 129: Guidance Document on Using Cytotoxicity Tests to Estimate Starting Doses for Acute Oral Systemic Toxicity Tests (available at: https://ntp.niehs.nih.gov/sites/default/files/iccvam/suppdocs/feddocs/oecd/oecd-gd129.pdf) Any surfactant identified chemicals should be assayed using the Depth of Migration (DM) test. Obtain Depth of Migration (DM) test cards and apply 30 μL of the negative control, positive control, and the triplicates of the test samples. Measure the distance traveled. These test samples all had a distanced traveled of 5.0 cm. Take the result from the Acute Toxicity test method and apply the distance traveled and the correction factor of 1.75. The false negative results will now correctly be predicted as a true positive. The false negative (FN) rate was significantly less when the distance migrated test was accounted for. The reduction of the false negative (FN) rate is attributed to the multiplication of the main test value by the depth of migration correction factor.

While various aspects and embodiments have been disclosed herein, other aspects and embodiments will be apparent to those skilled in the art.

The various aspects and embodiments disclosed herein are for purposes of illustration and are not intended to be limiting, with the true scope and spirit being indicated by the following claims.

One skilled in the art will appreciate that this and other processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

### BIBLIOGRAPHY

Choksi N., Lebrun S., Nguyen M., Daniel A., DeGeorge G., Willoughby J., Layton A., Lowther D., Merrill J., Matheson J., Barroso K., Yozzo K., Casey W., Allen D. (2020). “Validation of the OptiSafe™ eye irritation test”. Cutan Ocul Toxicol. 39(3):180-192. doi:10.1080/15569527.2020.1787431.
Draize J H., Woodard G., Calvery H O. (1944). “Methods for the study of irritation and toxicity of substances applied topically to the skin and mucous membranes”. J. Pharmacol. And Exp. Therapeutics. 82: 377-390.
EC. (2001). Commission Directive 2001/59/EC of 6 Aug. 2001 adapting to technical progress for the 28th time Council Directive 67/548/EEC on the approximation of the laws, regulations and administrative provisions relating to the classification, packaging and labelling of dangerous substances. Official Journal of the European Communities L225, 1-333.
EC. (2008a). Classification, Regulation and Packaging (CLP): Regulation (EC) No. 1272/2008. Available: [http://echa.europa.eu/web/guest/regulations/clp].
EC. (2008b). Regulation (EC) No 1272/2008 of the European Parliament and of the Council of 16 Dec. 2008 on Classification, Labelling and Packaging of substances and mixtures, amending and repealing Directives 67/548/EEC and 1999/45/EC, and amending Regulation (EC) No 1907/2006. Official Journal of the European Union L353, 1-1355.
H.R.2790. (2017). Humane Cosmetics Act 115th Congress (2018-2018). Rep. McSally, Martha. [R-AZ-2] (Introduced Mar. 5, 2014). https://www.congress.gov/bill/115th-congress/house-bill/2790/text.
Humane Society. (2017). Timeline: Cosmetics Testing on Animals: The Humane Society of the United States. [online] Available at: http://www.humanesociety.org/issues/cosmetic_testing/timelines/timeline-cosmetics-testing-on-animals.html [Accessed 27 Dec. 2017].
ICCVAM. (2009). Independent Scientific Peer Review Panel Report: Evaluation of the Validation Status of Alternative Ocular Safety Testing Methods and Approaches. Available at: http://iccvam.niehs.nih.gov/docs/ocutox_docs/OcularPRPRept2009. pdf
ICCVAM. (2010). “ICCVAM-Recommended Test Method Protocol: Hen's Egg Test-Chorioallantoic Membrane (HET-CAM) Test Method”. Available at: https://ntp. niehs.nih.gov/iccvam/docs/protocols/ivocular-hetcam.pdf
ICCVAM-NICEATM. (2013). “Short Time Exposure (STE) Test Method Summary Review Document”, National Toxicology Program. Available at: http://www.ntp.niehs.nih.gov/iccvam/docs/ocutox_docs/STE-SRD-NICEATM-508.pdf.
Kanter J. (2017). E.U. Bans Cosmetics With Animal-Tested Ingredients. [online] Nytimes.com. Available at: http://www.nytimes.com/2013/03/11/business/global/eu-to-ban-cosmetics-with-animal-tested-ingredients.html [Accessed 31 Dec. 2017].
Kay J H., Calandra J C. (1962). “Interpretation of eye irritation tests”. J. Soc. Cosmet. Chem. 13: 281-289.
Lebrun S., Choksi N., Daniel A., Allen D., Casey W. (2019). “Prevalidation of the OptiSafe Ocular Irritation Assay for the Detection of Ocular Corrosives”. SOT 2019 Annual Meeting.
Lebrun S., Nguyen L., Chavez S., Chan R., Le D., Nguyen M., Jester J V. (2020). “Same-chemical comparison of nonanimal eye irritation test methods: Bovine corneal permeability, EpiOcular™, isolated chicken eye, ocular Irritection®, OptiSafe™, and short time exposure”. Toxicol In Vitro. 2020: 105070. doi: 10.1016/j.tiv.2020.105070.
MatTek Corporation. (2021). Protocol: “EpiOcular™ Eye Irritation Test (OCL-200-EIT) For the prediction of acute ocular irritation of chemicals: Identification of chemicals not requiring classification and labeling for eye irritation or serious eye damage”. Available at: https://www.mattek.com/wp-content/uploads/OCL-200-EIT-Eye-Irritation-Test-Protocol-MK-24-007-0055_02_02_2021.pdf
OECD. (2018). “Test No. 438: Isolated Chicken Eye Test Method for Identifying i) Chemicals Inducing Serious Eye Damage and ii) Chemicals Not Requiring Classification for Eye Irritation or Serious Eye Damage”. OECD Guidelines for the Testing of Chemicals, Section 4, OECD Publishing, Paris, https://doi.org/10.1787/9789264203860-en.
OECD. (2019a). “Test No. 492: Reconstructed human Cornea-like Epithelium (RhCE) test method for identifying chemicals not requiring classification and labelling for eye irritation or serious eye damage”. OECD Guidelines for the Testing of Chemicals, Section 4, OECD Publishing, Paris, https://doi.org/10.1787/9789264242548-en.
OECD. (2019b). “Test No. 496: in vitro Macromolecular Test Method for Identifying Chemicals Inducing Serious Eye Damage and Chemicals not Requiring Classification for Eye Irritation or Serious Eye Damage. OECD Guidelines for the Testing of Chemicals, Section 4, OECD Publishing, Paris, https://doi.org/10.1787/970e5cd9-en
OECD. (2020a). “Test No. 437: Bovine Corneal Opacity and Permeability Test Method for Identifying i) Chemicals Inducing Serious Eye Damage and ii) Chemicals Not Requiring Classification for Eye Irritation or Serious Eye Damage”. OECD Guidelines for the Testing of Chemicals, Section 4, OECD Publishing, Paris, https://doi.org/i0.1787/9789264203846-en
OECD. (2020b). “Test No. 491: STE In Vitro Test Method for Identifying i) Chemicals Inducing Serious Eye Damage and ii) Chemicals Not Requiring Classification for Eye Irritation or Serious Eye Damage”. OECD Guidelines for the Testing of Chemicals, Section 4, OECD Publishing, Paris, https://doi.org/10.1787/9789264242432-en. S.697. (2016). Frank R. Lautenberg Chemical Safety for the 21st Century Act. 114th Congress (2015-2016) https://www.congress.gov/bill/114th-congress/senate-bill/697.
Senate Joint Resolution 22. (2014). Introduced by Senator Block. Mar. 24, 2014. http://leginfo.legislature.ca.gov/faces/billNavClient.xhtml?bill_id=201320140SJR22&search keywords=
UN (2011). United Nations Globally Harmonized System of Classification and Labelling of Chemicals (GHS), ST/SG/AC.10/30 Rev 4, Part 3 Health Hazards—Chapter 3.3 Serious eye damage/eye irritation. New York & Geneva: United Nations Publications. pp. 133-144. Available: [http://www.unece.org/trans/danger/publi/ghs/ghs_rev04/04files_e.html].
US EPA (1998). Health Effects Test Guideline, OPPTS 870.2400 Acute Eye Irritation. EPA 712-C-98-195.
US EPA (2003). Label Review Manual. 3rd Edition. EPA 735-B-03-001. US Environmental Protection Agency, Washington, DC, USA.
Ljubimov A V., Saghizadeh M. (2015). “Progress in corneal wound healing”. Prog Retin Eye Res. 49:17-45. doi: 10.1016/j.preteyeres.2015.07.002.
Torricelli A A., Santhanam A., Wu J., Singh V., Wilson S E. (2016). “The corneal fibrosis response to epithelial-stromal injury”. Exp Eye Res. 142:110-8. doi: 10.1016/j.exer.2014.09.012.
Mobaraki M., Abbasi R., Omidian Vandchali S., Ghaffari M., Moztarzadeh F., Mozafari M. (2019). “Corneal Repair and Regeneration: Current Concepts and Future Directions”. Front Bioeng Biotechnol. 7:135. doi: 10.3389/fbioe.2019.00135.
Dupps W J Jr., Wilson S E. (2006). “Biomechanics and wound healing in the cornea”. Exp Eye Res. 83(4):709-20. doi: 10.1016/j.exer.2006.03.015.

What is claimed is:

1. A method for predicting the toxicity of a test substance, the method comprising; applying a substance to be tested for toxicity to non-biological, synthetic material or mixture of materials with hydrophobic properties that approximate or model some of the properties of cell membranes and connective tissues, measuring the migration distance of the substance being tested on the synthetic test material, and using the measured value to predict the extent, level or degree of toxicity, irritation or tissue damage.

2. The method in claim 1, wherein the non-biological test material to measure migration comprises 10% or more of any one of the following materials, selected from polyvinyl chloride (CASRN 9002-86-2), polypropylene (CASRN 9003-07-0), polystyrene (CASRN 9003-53-6), acrylate (CASRN 25133-97-5), polyethylene (CASRN 9002-88-4), ethylene vinyl acetate (CASRN 24937-78-8), polyesters (CASRN 113669-97-9), polyurethanes (CASRN 9009-54-5).

3. The method of claim 2 where one or more of the materials is mixed, laminated or otherwise applied to additional materials that serves as solid supports, the solid support selected from cardboard, paper, plastic, glass, or similar.

4. The method of claim 1 where the non-biological material contains at least 20% polyvinyl chloride (CASRN 9002-86-2), styrene or acrylate.

5. The method of claim 1, wherein a dye is added to the material to be tested so as the migration distance is easily observed and/or measured.

6. The method of claim 1, wherein the distance migrated is determined by preprinted markers on the solid support.

7. The method of claim 1, wherein the distance migrated is measured with a ruler or similar measuring device.

8. The method of claims 1 and 2, wherein the migration distance is added, multiplied or otherwise used as a correction factor to a second measure of toxicity, and the migration corrected value is then used to predict a level or classification of toxicity.

9. The method of claim 8, where the first toxicity test is one of the following:
Bovine Corneal Opacity and Permeability (BCOP), EpiOcular™ Eye Irritation Test, Hen's Egg Test-Chorioallantoic Membrane (HET-CAM), Isolated Chicken Eye (ICE), Ocular Irritection®, OptiSafe Eye Irritation Test™, Short Time Exposure (STE), or similar.

10. A method for reducing false negative rates of nonanimal eye irritation tests, the method comprising: measuring the migration distance on a synthetic polymer, using the measured distance to correct for tested material migration distance; where corrected test includes a measure of cell viability, an in silico prediction, a macromolecular, organotypic or other toxicity test, and using this to improve the categorization or prediction according to established ocular irritancy classes, wherein the false negative rate is reduced compared to predicting the irritancy classification without using the migration distance measurement and correction.

11. The method of claim 10, wherein the established ocular irritancy classes are selected from a nonirritant, a minimal irritant, a mild irritant, and a severe irritant.

12. The method of claim 10, wherein the established ocular irritancy classes include GHS Categories NC, 2, 2B, 2A, and 1, or EPA Categories IV, III, II, and I.

13. A kit that includes the material described in claim 1 and the intended use of the kit is to be used alone or in combination with another toxicity test to improve the irritancy, tissue damage, or toxicity prediction.

14. A kit that includes the material described in claim 1 and the intended use of the kit is to be used alone or in combination with another toxicity test to improve the ocular irritancy, damage, or toxicity prediction.

15. A kit that includes the material described in claims 1 and 2, and the kit is used alone or in combination with another toxicity test to improve the ocular irritancy, damage or toxicity prediction.

16. A kit that includes the materials described in claims 1-3 used to predict the ocular toxicity or potential to irritate or damage the eye, alone or in combination with another toxicity test.

17. A method to improve the prediction of acute toxicity after oral ingestion, in which the depth measurement of claim 1 is used as a correction factor for a cell or tissue based in vitro test to identify substance that have acute or chronic toxicity, and the prediction is improved by the addition, multiplication or otherwise using as a correction factor the depth measurement of claim 1.

18. A method to improve the prediction of acute toxicity after oral ingestion, in which the depth measurement of claim 2 is used as a correction factor for a cell or tissue based in vitro test to identify substance that have acute or chronic toxicity, and the prediction is improved by the addition, multiplication or otherwise using as a correction factor the depth measurement of claim 2.

19. A method to test for toxicity that relates damage to macromolecules with loss of cell viability and the extent of measured macromolecular damage alone or in combination with claim 1 disclosure used to predict a decrease in cell viability and/or toxicity.

20. A kit to improve the prediction of acute toxicity after oral ingestion, in which the depth measurement of claim 1 is used as a correction factor for a cell or tissue based in vitro test to identify substance that have acute or chronic toxicity, and the prediction is improved by the addition, multiplication or otherwise using as a correction factor the depth measurement of claim 1.
